(19) United States
(12) Patent Application Publication
(10) Pub. No.: US 2005/0245949 A1
(43) Pub. Date: Nov. 3, 2005

(54) Thermokeratoplasty system with a guided probe tip

(76) Inventors: Paul R. Goth, Bristol, ME (US); Dorin Panescu, San Jose, CA (US); Steve Khalaj, Laguna Hills, CA (US)

Correspondence Address: IRELL & MANELLA LLP, 840 NEWPORT CENTER DRIVE, SUITE 400, NEWPORT BEACH, CA, 92660, US

(21) Appl. No.: 10/834,677

(22) Filed: Apr. 28, 2004

(51) Int. Cl. A61F 9/00

(52) U.S. Cl. 606/166

(57) ABSTRACT

A template that is used with a probe to perform a medical procedure on a cornea. One embodiment of the template includes one or more openings that are used to align the probe with locations of the cornea that are to be denatured with energy transferred by the probe. By way of example, the openings may be located at 6, 7 and 8 millimeters about the center of the cornea. The denatured spots may collectively decrease the radius of curvature of the cornea. The template may have a bottom surface that conforms to the shape of the cornea. The template may have a centering feature that is used to center the template on the cornea and a vacuum channel that maintains the position of the template. The template openings may have stop features that limit a penetration depth of the probe.

### BACKGROUND OF THE INVENTION

1. Field of the Invention

The present invention relates to a thermokeratoplasty system that is used to reshape a cornea.

2. Prior Art

Techniques for correcting vision have included reshaping the cornea of the eye. For example, myopic conditions can be corrected by cutting a number of small incisions in the corneal membrane. The incisions allow the corneal membrane to relax and increase the radius of the cornea. The incisions are typically created with either a laser or a precision knife. The procedure for creating incisions to correct myopic defects is commonly referred to as radial keratotomy and is well known in the art.

Radial keratotomy techniques generally make incisions that penetrate approximately 95% of the cornea. Penetrating the cornea to such a depth increases the risk of puncturing the Descemets membrane and the endothelium layer, and creating permanent damage to the eye. Additionally, light entering the cornea at the incision sight is refracted by the incision scar and produces a glaring effect in the visual field. The glare effect of the scar produces impaired night vision for the patient.

The techniques of radial keratotomy are only effective in correcting myopia. Radial keratotomy cannot be used to correct an eye condition such as hyperopia. Additionally, keratotomy has limited use in reducing or correcting an astigmatism. The cornea of a patient with hyperopia is relatively flat (large spherical radius). A flat cornea creates a lens system which does not correctly focus the viewed image onto the retina of the eye. Hyperopia can be corrected by reshaping the eye to decrease the spherical radius of the cornea. It has been found that hyperopia can be corrected by heating and denaturing local regions of the cornea. The denatured tissue contracts and changes the shape of the cornea and corrects the optical characteristics of the eye. The procedure of heating the corneal membrane to correct a patient's vision is commonly referred to as thermokeratoplasty.

U.S. Pat. No. 4,461,294 issued to Baron; U.S. Pat. No. 4,976,709 issued to Sand and PCT Publication WO 90/12618, all disclose thermokeratoplasty techniques which utilize a laser to heat the cornea. The energy of the laser generates localized heat within the corneal stroma through photonic absorption. The heated areas of the stroma then shrink to change the shape of the eye.

Although effective in reshaping the eye, the laser based systems of the Baron, Sand and PCT references are relatively expensive to produce, have a non-uniform thermal conduction profile, are not self limiting, are susceptible to providing too much heat to the eye, may induce astigmatism and produce excessive adjacent tissue damage, and require long term stabilization of the eye. Expensive laser systems increase the cost of the procedure and are economically impractical to gain widespread market acceptance and use.

Additionally, laser thermokeratoplasty techniques non-uniformly shrink the stroma without shrinking the Bowmans layer. Shrinking the stroma without a corresponding shrinkage of the Bowmans layer, creates a mechanical strain in the cornea. The mechanical strain may produce an undesirable reshaping of the cornea and probable regression of the visual acuity correction as the corneal lesion heals. Laser techniques may also perforate Bowmans layer and leave a leucoma within the visual field of the eye.

U.S. Pat. Nos. 4,326,529 and 4,381,007 issued to Doss et al, disclose electrodes that are used to heat large areas of the cornea to correct for myopia. The electrode is located within a sleeve that suspends the electrode tip from the surface of the eye. An isotropic saline solution is irrigated through the electrode and aspirated through a channel formed between the outer surface of the electrode and the inner surface of the sleeve. The saline solution provides an electrically conductive medium between the electrode and the corneal membrane. The current from the electrode heats the outer layers of the cornea. Heating the outer eye tissue causes the cornea to shrink into a new radial shape. The saline solution also functions as a coolant which cools the outer epithelium layer.

The saline solution of the Doss device spreads the current of the electrode over a relatively large area of the cornea. Consequently, thermokeratoplasty techniques using the Doss device are limited to reshaped corneas with relatively large and undesirable denatured areas within the visual axis of the eye. The electrode device of the Doss system is also relatively complex and cumbersome to use.

“A Technique for the Selective Heating of Corneal Stroma” Doss et al., Contact & Intraoccular Lens Medical Jrl., Vol. 6, No. 1, pp. 13-17, January`-March, 1980, discusses a procedure wherein the circulating saline electrode (CSE) of the Doss patent was used to heat a pig cornea. The electrode provided 30 volts r.m.s. for 4 seconds. The results showed that the stroma was heated to 70° C. and the Bowman's membrane was heated 45° C., a temperature below the 50-55° C. required to shrink the cornea without regression.

“The Need For Prompt Prospective Investigation” McDonnell, Refractive & Corneal Surgery, Vol. 5, January/February, 1989 discusses the merits of corneal reshaping by thermokeratoplasty techniques. The article discusses a procedure wherein a stromal collagen was heated by radio frequency waves to correct for a keratoconus condition. As the article reports, the patient had an initial profound flattening of the eye followed by significant regression within weeks of the procedure.

“Regression of Effect Following Radial Thermokeratoplasty in Humans” Feldman et al., Refractive and Corneal Surgery, Vol. 5, September/October, 1989, discusses another thermokeratoplasty technique for correcting hyperopia. Feldman inserted a probe into four different locations of the cornea. The probe was heated to 600° C. and was inserted into the cornea for 0.3 seconds. Like the procedure discussed in the McDonnell article, the Feldman technique initially reduced hyperopia, but the patients had a significant regression within 9 months of the procedure.

Refractec, Inc. of Irvine Calif., the assignee of the present application, has developed a system to correct hyperopia and presbyopia with a thermokeratoplasty probe that is connected to a console. The probe includes a tip that is inserted into the stroma layer of a cornea. Electrical current provided by the console flows through the eye to denature the collagen tissue within the stroma. The process of inserting the probe tip and applying electrical current can be repeated in a circular pattern about the cornea. The procedure of applying RF energy through a probe tip to denature corneal tissue is taught by Refractec under the service marks CONDUCTIVE KERATOPLASTY and CK.

In a CK procedure probe tip placement is initially marked with a corneal marker. The doctor must then manually push the probe tip into the marked locations to deliver RF energy. Manual placement and insertion of the tip allows for human error. The surgeon may insert the probe tip too far, or not far enough, into the cornea. Lateral placement error may also occur during insertion of the probe. Additionally, it has been found that more satisfactory results are obtained when the tip enters the cornea perpendicular to the corneal surface. Insertion at an oblique angle may create an undesirable thermal gradient in the cornea during application of RF-energy.

### BRIEF SUMMARY OF THE INVENTION

A template that is used with a probe to perform a medical procedure on a cornea. The template has an opening that is used to align the probe with a spot of the cornea and a bottom surface that conforms with the shape of the cornea.

### BRIEF DESCRIPTION OF THE DRAWINGS

FIG. 1 is a perspective view of a thermokeratoplasty system;

FIG. 2 is a graph showing a waveform that is provided by a console of the system;

FIG. 3A is an enlarged view of a tip inserted into a cornea;

FIG. 3B is an enlarged view showing a spring loaded actuator that pushes the tip into the cornea;

FIG. 4 is a top view showing a pattern of denatured areas of the cornea;

FIG. 5 is a cross-sectional view of an alternate embodiment of a probe tip;

FIG. 6 is a cross-sectional view of an alternate embodiment of a probe tip;

FIG. 7 is a cross-sectional view of a template;

FIG. 8 is a perspective view showing a template coupled to a lid speculum;

FIG. 9 is a template that can guide a tip loaded sleeve;

FIG. 10 is a cross-sectional view of another embodiment of the template;

FIG. 11 is a top perspective view of an alternate embodiment of a template;

FIG. 12 is a cross-sectional view of the template shown in FIG. 11;

FIG. 13 is another embodiment of a template with a handle.

### DETAILED DESCRIPTION

Disclosed is a template that is used with a probe to perform a medical procedure on a cornea. One embodiment of the template includes one or more openings that are used to align the probe with locations of the cornea that are to be denatured with energy transferred by the probe. By way of example, the openings may be located at 6, 7 and 8 millimeters about the center of the cornea. The denatured spots may collectively decrease the radius of curvature of the cornea. The template may have a bottom surface that conforms to the shape of the cornea. The template may have a centering feature that is used to center the template on the cornea and a vacuum channel that maintains the position of the template. The template openings may have stop features that limit a penetration depth of the probe.

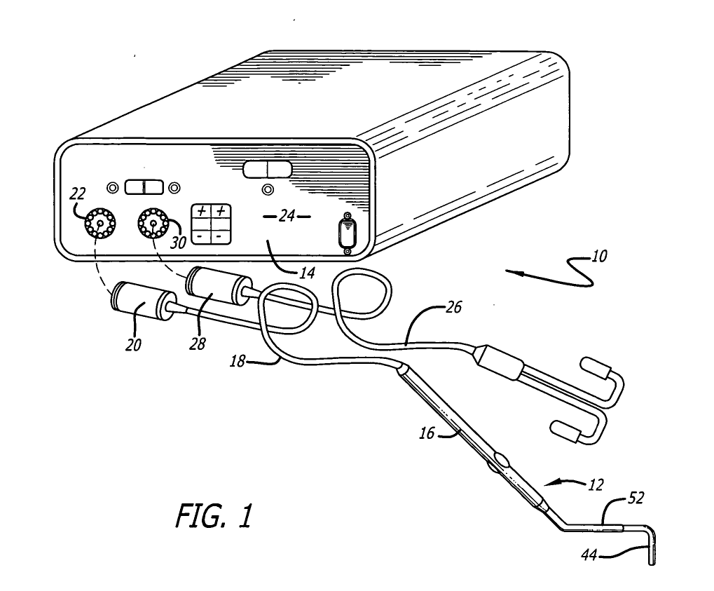

Referring to the drawings more particularly by reference numbers, FIG. 1 shows a thermokeratoplasty electrode system 10 of the present invention. The system 10 includes an electrode probe 12 coupled to a console 14. The console 14 contains a power supply that can deliver electrical power to the probe 12. The probe 12 has a hand piece 16 and wires 18 that couple the probe electrode to a connector 20 that plugs into a mating receptacle 22 located on the front panel 24 of the console 14. The hand piece 16 may be constructed from a non-conductive material.

The system 10 also includes a return element 26 that is in contact with the patient to provide a return path for the electrical current provided by the console 14 to the probe 12. The return element 26 has a connector 28 that plugs into a mating receptacle 30 located on the front panel 24 of the console 14. By way of example, the return element 26 may be a lid speculum that is used to maintain the patient's eyelids in an open position while providing a return path for the electrical current.

The console 14 provides a predetermined amount of energy, through a controlled application of power for a predetermined time duration. The console 14 may have manual controls that allow the user to select treatment parameters such as the power and time duration. The console 14 can also be constructed to provide an automated operation. The console 14 may have monitors and feedback systems for measuring physiologic tissue parameters such as tissue impedance, tissue temperature and other parameters, and adjust the output power of the radio frequency amplifier to accomplish the desired results.

In one embodiment, the console provides voltage limiting to prevent arcing. To protect the patient from overvoltage or overpower, the console 14 may have an upper voltage limit and/or upper power limit which terminates power to the probe when the output voltage or power of the unit exceeds a predetermined value.

The console 14 may also contain monitor and alarm circuits which monitors physiologic tissue parameters such as the resistance or impedance of the load and provides adjustments and/or an alarm when the resistance/impedance value exceeds and/or falls below predefined limits. The adjustment feature may change the voltage, current, and/or power delivered by the console such that the physiological parameter is maintained within a certain range. The alarm may provide either an audio and/or visual indication to the user that the resistance/impedance value has exceeded the outer predefined limits. Additionally, the unit may contain a ground fault indicator, and/or a tissue temperature monitor. The front panel 24 of the console 14 typically contains meters and displays that provide an indication of the power, frequency, etc., of the power delivered to the probe.

The console 14 may deliver a radiofrequency (RF) power output in a frequency range of 100 KHz-5 MHz. In the preferred embodiment, power is provided to the probe at a frequency in the range of 350 KHz. The console 14 is designed so that the power supplied to the probe 12 does not exceed a certain upper limit of up to several watts. Preferably the console is set to have an upper power limit of 1.2 watts (W). The time duration of each application of power to a particular corneal location can be up to several seconds but is typically set between 0.1-1.0 seconds. The unit 14 is preferably set to deliver approximately 0.6 W of power for 0.6 seconds.

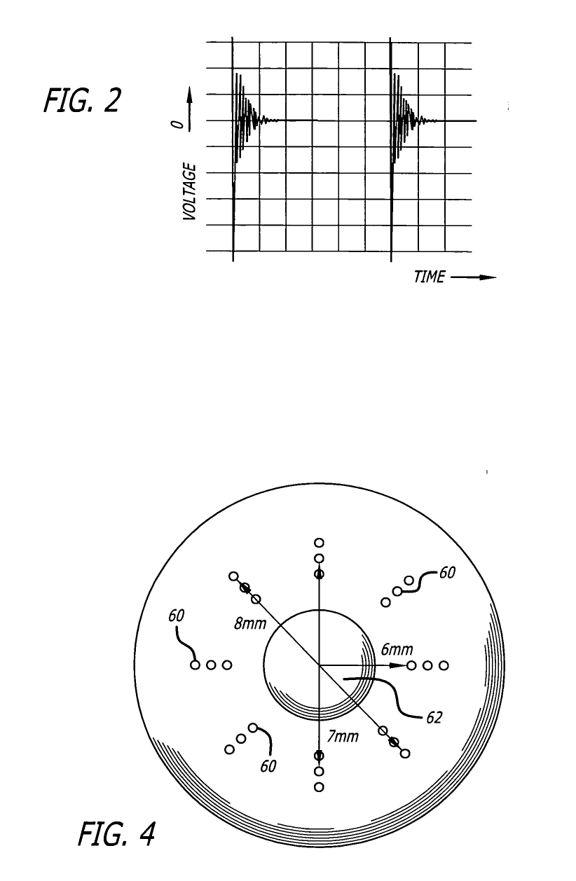

FIG. 2 shows a typical voltage waveform that is delivered by the probe 12 to the cornea. Each pulse of energy delivered by the probe 12 may be a highly damped sinusoidal waveform, typically having a crest factor (peak voltage/RMS voltage) greater than 5:1. Each highly damped sinusoidal waveform is repeated at a repetitive rate. The repetitive rate may range between 4-12 KHz and is preferably set at 7.5 KHz. Although a damped waveform is shown and described, other waveforms, such as continuous sinusoidal, amplitude, frequency or phase-modulated sinusoidal, etc. can be employed.

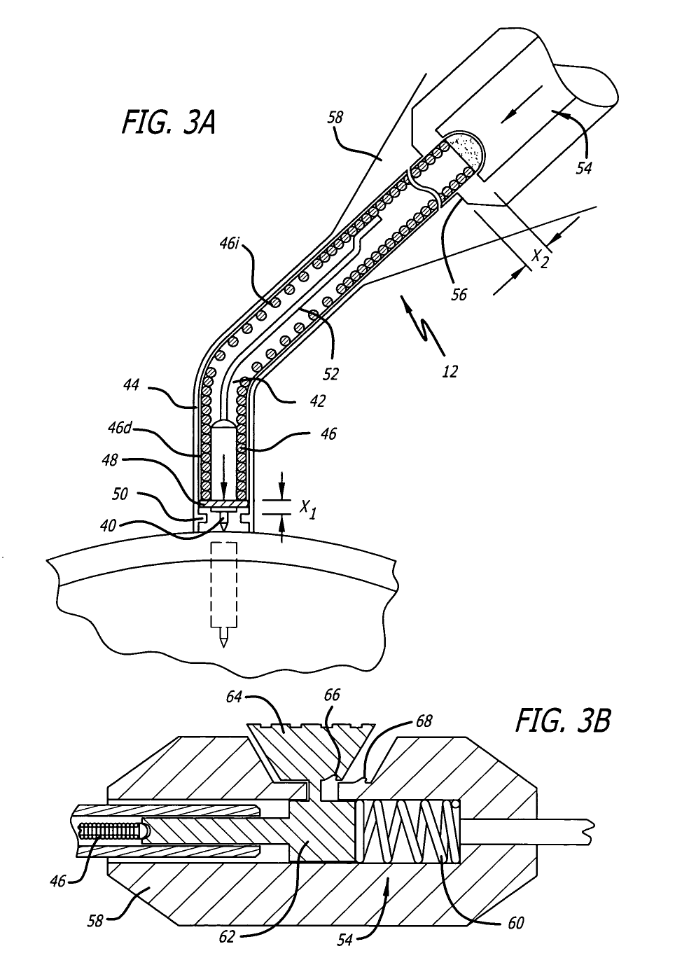

FIG. 3A, shows an embodiment of a probe 12 with a tip 40 located within an inner channel 42 of a sleeve 44. The probe 12 may be attached to a return spring 46 that biases the tip 40 away the cornea. The tip 40 includes a collar 48 that engages a stop 50 of the sleeve to limit the insertion depth of the tip 40 into the cornea. The tip 40 may be connected to the spring 46 by a wire 52. The spring 46 and wire 52 are electrically connected to the console (not shown). The distal portion of the spring 46d is attached to the sleeve 44.

The tip 40 can be actuated through movement of an actuator 54. Movement of the actuator 54 compresses an intermediate portion of the spring 46i which moves the wire 52 and the tip 40. The actuator movement is limited by a stop 56 of a housing 58. The movement of the actuator 54, shown as X₂, is greater than the movement of the collar 48 into stop 50, shown as X₁, so that there is an additional compression of the spring 46.

FIG. 3B shows an embodiment of an actuator 54 that drives the tip 40 into the cornea. The actuator 54 may have an action spring 60 that is coupled to a plunger 62. The plunger 62 is coupled to the proximal portion of the spring 46. The plunger 62 may have a button 64 with a groove 66 that can receive a detent 68 of the housing 58.

In operation the surgeon moves the button 64 so that the detent 68 locks into the groove 66. In this position the action spring 60 is compressed and retains a potential energy. The surgeon may then push the button 64 to release the detent 68 wherein the action spring 60 provides a driving force, thereby kinetic energy, that moves the plunger 62, spring 46 and tip 40. The actuator 54 provides a means for repetitively applying a puncture force to insert the tip into the cornea without reliance on manual insertion by the surgeon.

The probe 12 provides a current to the cornea through the tip 40. The current denatures the collagen tissue of the stroma. Because the tip 40 is inserted into the stroma it has been found that a power no greater than 1.2 watts for a time duration no greater than 1.0 seconds will adequately denature the corneal tissue to provide optical correction of the eye. However, other power and time limits, in the range of several watts and seconds, respectively, can be used to effectively denature the corneal tissue. Inserting the tip 40 into the cornea provides improved repeatability over probes placed into contact with the surface of the cornea, by reducing the variances in the electrical characteristics of the epithelium and the outer surface of the cornea.

FIG. 4 shows a pattern of denatured areas 60 that have been found to correct hyperopic or presbyopic conditions. A circle of 8, 16, or 24 denatured areas 60 are created about the center of the cornea, outside the visual axis portion 62 of the eye. The visual axis has a nominal diameter of approximately 5 millimeters. It has been found that 16 denatured areas provide the most corneal shrinkage and less post-op astigmatism effects from the procedure. The circle of denatured areas typically have a diameter between 6-8 mm, with a preferred diameter of approximately 7 mm. If the first circle does not correct the eye deficiency, the same pattern may be repeated, or another pattern of 8 denatured areas may be created within a circle having a diameter of approximately 6.0-6.5 mm either in line or overlapping. The assignee of the present application provides instructional services to educate those performing such procedures under the service marks CONDUCTIVE KERATOPLASTY and CK.

The exact diameter of the pattern may vary from patient to patient, it being understood that the denatured spots should preferably be formed in the non-visionary portion 62 of the eye. Although a circular pattern is shown, it is to be understood that the denatured areas 60 may be located in any location and in any pattern. In addition to correcting for hyperopia, the present invention may be used to correct astigmatic conditions. For correcting astigmatic conditions, the denatured areas are typically created at the end of the astigmatic flat axis. The present invention may also be used to correct procedures that have overcorrected for a myopic condition.

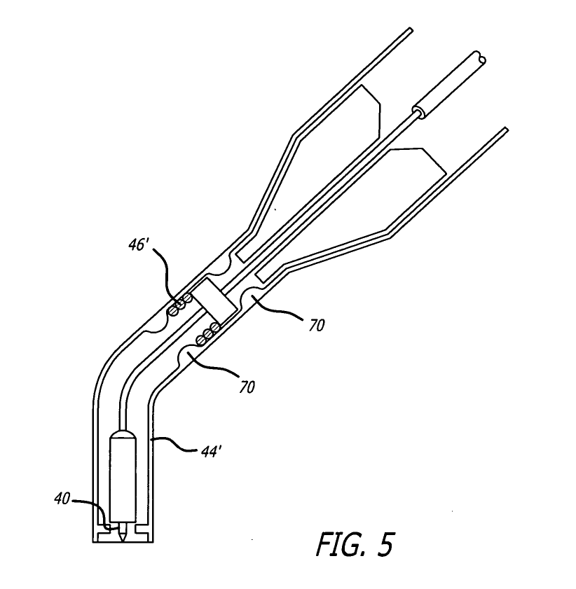

FIG. 5 shows an alternate embodiment of a probe wherein the spring 46′ is captured between two inner protrusions 70 of the sleeve 44′.

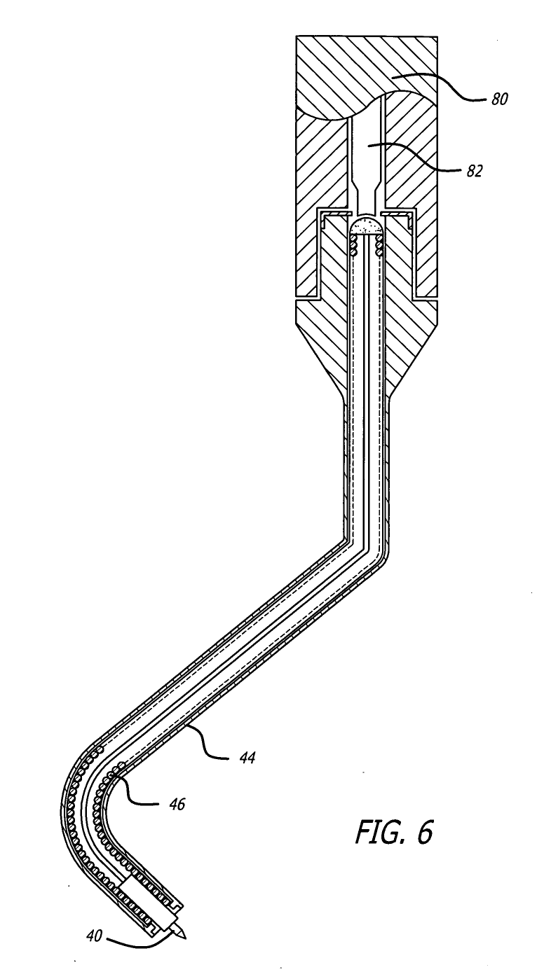

FIG. 6 shows an embodiment of a probe with an actuator 80 that drives an actuator pin 82 to move the tip 40. By way of example, the actuator 80 may be a pneumatic, hydraulic, electric, electromagnetic or piezoelectric device. Additionally, the actuator 80 may be constructed to move the tip 40 both into and out of the cornea. The actuator 80 may be connected to the console 24 which automatically operates a routine of inserting the tip 40 into the cornea, applying energy, and pulling the tip out of the cornea. The actuator 80 may also provide a consistent insertion force for each application of energy.

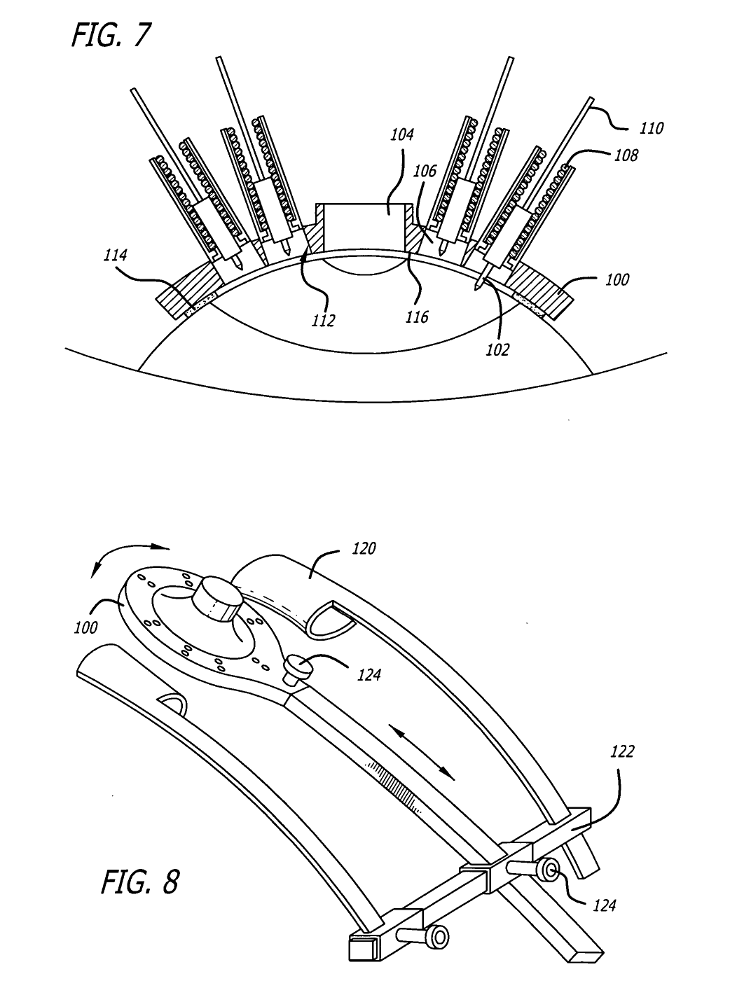

FIG. 7 shows an embodiment of a template 100 that can align a plurality of probe tips 102 relative to a cornea. The template 100 may have a centering feature such as a centering aperture 104. The centering aperture 104 can be used to align the template 100 onto the cornea. By way of example, a ring light (not shown) can be used to project a ring of light onto the cornea. The surgeon can then align the centering aperture 104 with the ring of light to center the template 100.

The probe tips 102 may extend through corresponding openings 106 in the template 100. The openings 106 may be located in circular patterns that are 6, 7 and 8 millimeters about the center of the cornea. The openings 106 align the probe tips 102 to create the circular patterns shown in FIG. 4.

The probe tips 102 may have springs 108, electrical wires 110 and stop features 112 that, return the tips and/or provide a consistent insertion force, provide RF electrical power, and limit the insertion depth of the tips 102, respectively. The template 100 may be held in place by suction cups 114.

The template 100 preferably has a compound first surface 116 that conforms to the shape of the cornea. It is preferable to construct the openings 106 so that the longitudinal axis of each probe tip 102 is perpendicular to the surface of the cornea. This increases the likelihood that the tips 102 will be inserted into the cornea in a direction that is perpendicular to the corneal surface. It is believed that a perpendicular insertion will create a more uniform thermal gradient and a more desirable effect on the cornea. The tips 102 can be inserted into the cornea manually or by an actuator. Additionally, the tips 102 can be inserted into the cornea and apply RF energy, either sequentially, or simultaneously.

FIG. 8 shows an embodiment where the template 100 is coupled to a lid speculum 120 by a frame 122. The position of the template 100 can be adjusted through screws 124 in the directions indicated by the arrows. The frame 122 allows the surgeon to hold one hand piece to place both the lid speculum 120 and the template 100 onto the patient.

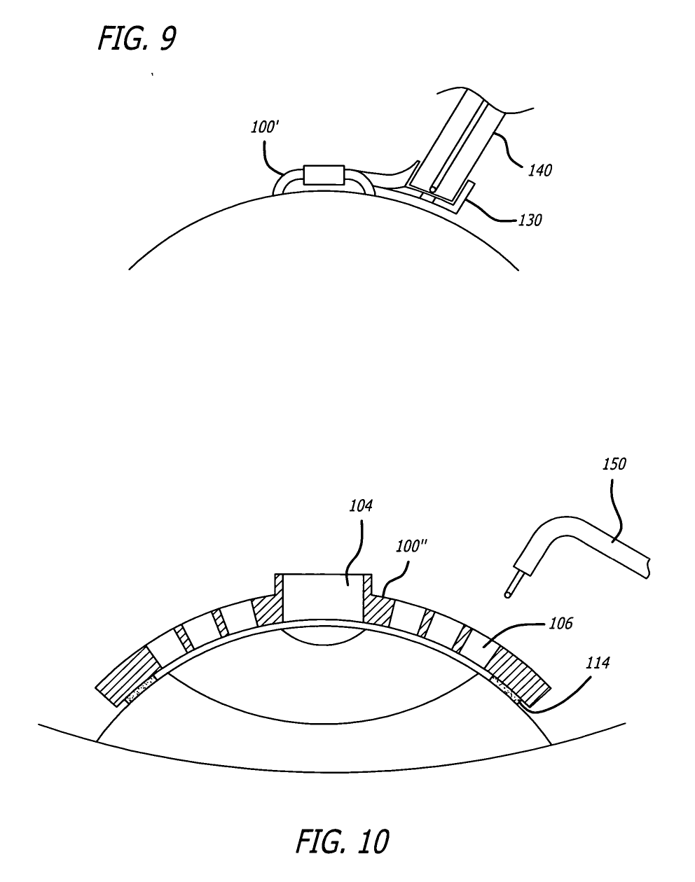

FIG. 9 is an alternate embodiment of a template 100′ with a seat 130 that can receive and align a sleeve type probe 140 that is the same or similar to the sleeve concepts shown in FIGS. 3, 5 and 6.

FIG. 10 is an alternate embodiment of a template 100″ that contains openings 106 that align a hand held probe 150. The template 100″ could be used with existing probe tips sold by the assignee, Refractec, under the trademark KERATOPLAST.

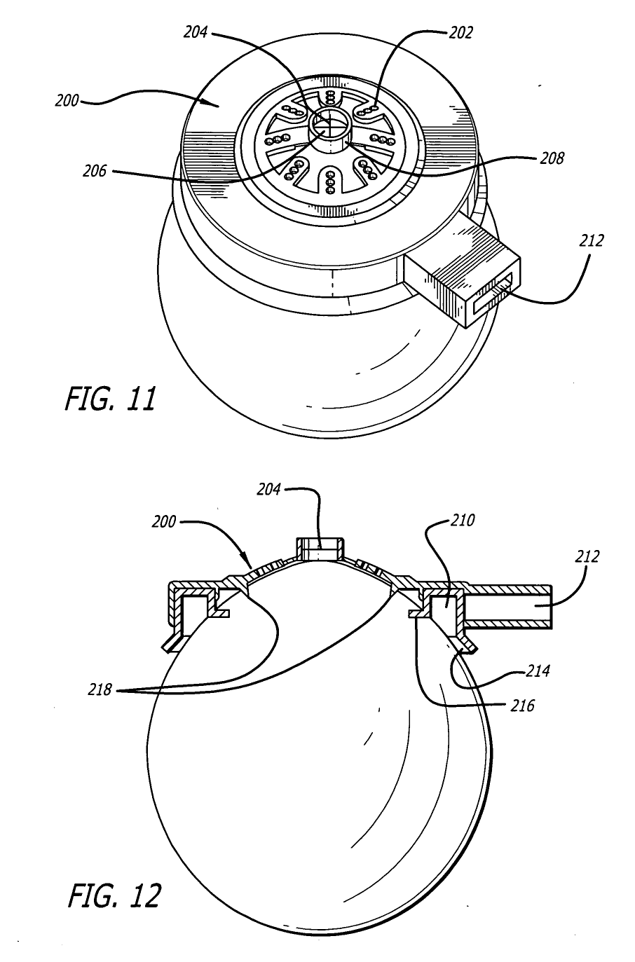

FIGS. 11 and 12 show another embodiment of a template 200 that can create a vacuum to maintain the position of the plate 200. The template 200 may be constructed from an optically transparent material so that the surgeon can see the cornea through the plate. The plate 200 may have a plurality of openings 202 that can guide an electrode probe (not shown). By way of example, the openings 202 may be located in circular patterns at 6, 7 and 8 millimeters.

The template 200 may include a reticle 204 formed within a center opening 206 of an inner ring 208. The reticle 204 can be used by the surgeon to center the template 200 onto the cornea.

As shown in FIG. 12, the template 200 may include an inner channel 210 that is in fluid communication with a vacuum port 212. The vacuum port 212 is connected to a source of vacuum (not shown) that can create a vacuum pressure within the inner channel 210. The vacuum pressure maintains the position of the template 200 onto the cornea.

The inner channel 210 may be sealed by an elastomeric seal 214 and an elastic flap 216. The template 200 may also have a protrusion 218 that creates an additional force to maintain the position of the template 200. The protrusion 218 may have different shapes and configurations. In general, the protrusion 218 may slightly extend into the cornea and/or provide frictional forces to maintain the position of the template 200. The protrusions and/or vacuum pressure may exert a pressure on the cornea to stiffen the corneal tissue. Stiffening the cornea decreases tissue flexibility and allows the probe tip to more easily puncture the cornea.

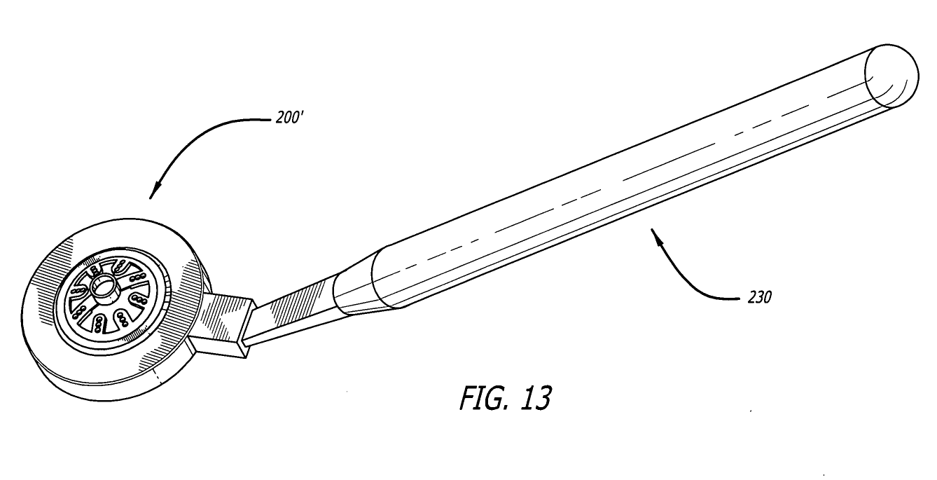

FIG. 13 shows an alternate embodiment of a template 200′ with a handle 230. Although not shown the templates 200 and 200′ may have the integrated probes, springs, actuators, etc. in the embodiment shown in FIGS. 3, 5, 6 and 7. The surgeon may push down on the handle 230 to maintain the position of the template 200′. Care should be taken to avoid applying an excessive pressure that cuts-off the supply of blood to the eye.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

For example, although the delivery of radio frequency energy is described, it is to be understood that other types of non-thermal energy such as direct current (DC), microwave, ultrasonic and light can be transferred into the cornea. Non-thermal energy does not include the concept of heating a tip that had been inserted or is to be inserted into the cornea.

By way of example, the console can be modified to supply energy in the microwave frequency range or the ultrasonic frequency range. By way of example, the probe may have a helical microwave antenna with a diameter suitable for corneal delivery. The delivery of microwave energy could be achieved with or without corneal penetration, depending on the design of the antenna. The system may modulate the microwave energy in response to changes in the characteristic impedance.

For ultrasonic application, the probe would contain a transducer that is driven by the console and mechanically oscillates the tip. The system could monitor acoustic impedance and provide a corresponding feedback/regulation scheme For application of light the probe may contain some type of light guide that is inserted into the cornea and directs light into corneal tissue. The console would have means to generate light, preferably a coherent light source such as a laser, that can be delivered by the probe. The probe may include lens, waveguide and a photodiode that is used sense reflected light and monitor variations in the index of refraction, birefringence index of the cornea tissue as a way to monitor physiological changes and regulate power.

1. An apparatus that is used with a probe in a medical procedure on a cornea with a radial shape, comprising:
a template with a bottom surface that conforms to the radial shape of the cornea and an openings that guides the probe into a spot on the cornea.

2. The apparatus of claim 1, wherein said bottom surface has a compound curvature.

3. The apparatus of claim 1, wherein said template has a plurality of openings.

4. The apparatus of claim 3, wherein said openings are arranged in a circular pattern.

5. The apparatus of claim 3, wherein said openings are arranged in a plurality of circular patterns having diameters at 6, 7 and 8 millimeters.

6. The apparatus of claim 3, further comprising a plurality of probes that are coupled to said template and extend though said openings.

7. The apparatus of claim 1, wherein said template includes a vacuum channel.

8. The apparatus of claim 1, wherein said template includes a sealing ring.

9. The apparatus of claim 1, wherein said template includes a protrusion that extends into the cornea.

10. The apparatus of claim 1, wherein said template includes an alignment feature.

11. The apparatus of claim 10, wherein said alignment feature includes a reticle within a center opening.

12. The apparatus of claim 1, wherein the probe is coupled to said template and extends through said opening of said template.

13. The apparatus of claim 12, further comprising a spring coupled to the probe and said template.

14. The apparatus of claim 12, further comprising an actuator coupled to the probe.

15. The apparatus of claim 12, wherein said template includes a stop that limits a penetration depth of the probe.

16. The apparatus of claim 1, further comprising a lid speculum coupled to said template.

17. The apparatus of claim 12, further comprising a console that provides radio frequency power to the probe.

18. The apparatus of claim 14, wherein said template includes a handle.

19. An apparatus that is used with a probe in a medical procedure on a cornea, comprising:
a template with a vacuum channel and an opening that is used to align the probe with a spot on the cornea.

20. The apparatus of claim 19, wherein said template has a bottom surface with a compound curvature.

21. The apparatus of claim 19, wherein said template has a plurality of openings.

22. The apparatus of claim 21, wherein said openings are arranged in a circular pattern.

23. The apparatus of claim 21, wherein said openings are arranged in a plurality of circular patterns having diameters at 6, 7 and 8 millimeters.

24. The apparatus of claim 23, further comprising a plurality of probes that are coupled to said template and extend though said openings.

25. The apparatus of claim 19, wherein said template includes a sealing ring.

26. The apparatus of claim 19, wherein said template has a protrusion that extends into the cornea.

27. The apparatus of claim 19, wherein said template includes an alignment feature.

28. The apparatus of claim 27, wherein said alignment feature includes a reticle within a center opening.

29. The apparatus of claim 19, wherein said template includes a handle.

30. An apparatus that is used with a probe in a medical procedure on a cornea, comprising:
a template with a protrusion that maintains a position of said template on the cornea and an opening that is used to align the probe with a spot on the cornea.

31. The apparatus of claim 30, wherein template has a bottom surface with a compound curvature.

32. The apparatus of claim 30, wherein said template has a plurality of openings.

33. The apparatus of claim 32, wherein said openings are arranged in a circular pattern.

34. The apparatus of claim 32, wherein said openings are arranged in a plurality of circular patterns having diameters at 6, 7 and 8 millimeters.

35. The apparatus of claim 32, further comprising a plurality of probes that are coupled to said template and extend though said openings.

36. The apparatus of claim 30, wherein said template includes a sealing ring.

37. The apparatus of claim 30, wherein said template includes an alignment feature.

38. The apparatus of claim 37, wherein said alignment feature includes a reticle within a center opening.

39. The apparatus of claim 30, wherein said template includes a handle.

40. An apparatus that is used with a probe in a medical procedure on a cornea, comprising:
a template with means for maintaining a position of said template on the cornea and an opening that is used to align the probe with a spot on the cornea.

41. The apparatus of claim 40, wherein said means includes a vacuum channel.

42. The apparatus of claim 40, wherein said means includes a protusion.

43. The apparatus of claim 40, wherein template has a bottom surface with a compound curvature.

44. The apparatus of claim 40, wherein said template has a plurality of openings.

45. The apparatus of claim 44, wherein said openings are arranged in a circular pattern.

46. The apparatus of claim 44, wherein said openings are arranged in a plurality of circular patterns having diameters at 6, 7 and 8 millimeters.

47. The apparatus of claim 44, further comprising a plurality of probes that are coupled to said template and extend though said openings.

48. The apparatus of claim 41, wherein said template includes a sealing ring.

49. The apparatus of claim 40, wherein said template includes alignment means for aligning said template onto the cornea.

50. The apparatus of claim 49, wherein said alignment means includes a reticle within a center opening.

51. The apparatus of claim 40, wherein said template includes a handle.

52. An apparatus that is used with a probe in a medical procedure on a cornea, comprising:
a template with a centering feature and an opening that is used to align the probe with a spot on the cornea.

53. The apparatus of claim 52, wherein said template has a bottom surface with a compound curvature.

54. The apparatus of claim 52, wherein said template has a plurality of openings.

55. The apparatus of claim 54, wherein said openings are arranged in a circular pattern.

56. The apparatus of claim 54, wherein said openings are arranged in a plurality of circular patterns having diameters at 6, 7 and 8 millimeters.

57. The apparatus of claim 54, further comprising a plurality of probes that are coupled to said template and extend though said openings.

58. The apparatus of claim 52, wherein said template includes a sealing ring.

59. The apparatus of claim 52, wherein said template has a protrusion that extends into the cornea.

60. The apparatus of claim 52, wherein said alignment feature includes a reticle within a center opening.

61. The apparatus of claim 52, wherein said template includes a handle.

62. An apparatus that is used with a probe in a medical procedure on a cornea, comprising:
a template with an opening that is used to align the probe with a spot on the cornea and alignment means for aligning said template with the cornea.

63. The apparatus of claim 62, wherein said template has a bottom surface with a compound curvature.

64. The apparatus of claim 62, wherein said template has a plurality of openings.

65. The apparatus of claim 64, wherein said openings are arranged in a circular pattern.

66. The apparatus of claim 64, wherein said openings are arranged in a plurality of circular patterns having diameters at 6, 7 and 8 millimeters.

67. The apparatus of claim 64, further comprising a plurality of probes that are coupled to said template and extend though said openings.

68. The apparatus of claim 62, wherein said template includes a sealing ring.

69. The apparatus of claim 62, wherein said template has a protrusion that extends into the cornea.

70. The apparatus of claim 62, wherein said alignment means includes a reticle within a center opening.

71. The apparatus of claim 62, wherein said template includes a handle.

72. A method for performing a medical procedure on a cornea, comprising:
aligning a template onto a cornea;
maintaining a position of the template on the cornea;
pushing a probe through an opening of the template into contact with the cornea; and,
delivering an energy to the cornea through the probe to denature corneal tissue.

73. The method of claim 72, wherein the template is aligned through a center opening.

74. The method of claim 72, wherein a portion of the probe is inserted into the cornea.

75. The method of claim 72, wherein a spring pulls the probe out of contact with the cornea.

76. The method of claim 72, wherein a spring pushes the probe in contact with the cornea.

77. The method of claim 72, wherein the probe is pushed into the cornea until engaging a stop.

78. The method of claim 72, wherein the position of the template is maintained with a vacuum pressure.

79. The method of claim 72, wherein the vacuum pressure tightens the cornea.

80. The method of claim 72, wherein the position of the template is maintained with a protrusion.

81. The method of claim 72, wherein the template is aligned through a reticle.

82. An apparatus that is used to perform a medical procedure on a cornea, comprising:
a probe; and,
an actuator that is coupled to said probe.

83. The apparatus of claim 82, wherein said actuator includes an action spring.

84. The apparatus of claim 82, further comprising a return spring coupled to said probe.

85. The apparatus of claim 82, wherein said actuator includes an electromagnetic device.

86. The apparatus of claim 82, wherein said actuator includes a pneumatic device.

87. The apparatus of claim 82, wherein said actuator includes a piezoelectric device.

88. An apparatus that is used to perform a medical procedure on a cornea, comprising:
a probe; and,
actuator means for moving said probe toward the cornea.

89. The apparatus of claim 88, wherein said actuator means includes an action spring.

90. The apparatus of claim 88, where said actuator means moves said probe out of contact with the cornea.

91. The apparatus of claim 90, further comprising a return spring coupled to said probe.

92. The apparatus of claim 88, wherein said actuator means includes an electromagnetic device.

93. The apparatus of claim 88, wherein said actuator means includes a pneumatic device.

94. The apparatus of claim 88, wherein said actuator means includes a piezoelectric device.

95. A method for performing a medical procedure on a cornea, comprising:
actuating an actuator to push a probe into contact with the cornea; and,
delivering an energy to the cornea through the probe to denature corneal tissue.

96. The method of claim 95, wherein a portion of the probe is inserted into the cornea.

97. The method of claim 95, wherein a spring pulls the probe out of contact with the cornea.

98. The method of claim 95, wherein the probe is pushed into the cornea until engaging a stop.
